(19) United States
(12) Patent Application Publication
(10) Pub. No.: US 2014/0142571 A1
(43) Pub. Date: May 22, 2014

(54) Liquid Crystalline Polymer Composition for Melt-Extruded Substrates

(71) Applicant: Ticona LLC, Florence, KY (US)

(72) Inventors: Paul C. Yung, Cincinnati, OH (US); James P. Shepherd, Independence, KY (US)

(73) Assignee: Ticona LLC, Florence, KY (US)

(21) Appl. No.: 14/082,394

(22) Filed: Nov. 18, 2013

Related U.S. Application Data
(60) Provisional application No. 61/728,858, filed on Nov. 21, 2012.
(60) Provisional application No. 61/778,929, filed on Mar. 13, 2013.

(51) Int. Cl. A61L 29/06; A61B 18/14; H05K 1/09; C08K 3/22

(52) U.S. Cl. CPC A61L 29/06; C08K 3/22; A61B 18/1492; H05K 1/09
USPC 606/41; 524/407; 428/36.9; 174/257

(57) ABSTRACT

A polymer composition that can be readily melt-extruded into a shaped three-dimensional substrate (e.g., tube) and also applied with a conductive element using a laser direct structuring (“LDS”) process. In this regard, the composition contains a thermotropic liquid crystalline polymer and a laser activatable additive. The specific nature of the polymer and relative concentration of the polymer and additive are selectively controlled so that the resulting composition can possess both a relatively high melt viscosity and melt strength.

### RELATED APPLICATIONS

The present application claims priority to U.S. Provisional Application Ser. Nos. 61/728,858 (filed on Nov. 21, 2012) and 61/778,929 (filed on Mar. 13, 2013), which are incorporated herein in their entirety by reference thereto.

### BACKGROUND OF THE INVENTION

A wide variety of different products employ “circuitized substrates” in which a plurality of conductive elements are formed on a surface of a plastic material. One such product, for instance, is a catheter used to examine, diagnose, and treat while positioned at a specific location within a body. For example, “catheter ablation” employs a catheter to convey an electrical stimulus to a selected location within the body to create tissue necrosis. Likewise, “mapping” employs a catheter to monitor various forms of electrical activity in the body. Conventional ablation and mapping catheters are labor-intensive to assemble and require, for instance, individually brazing each electrode to a conductor, puncturing holes into the catheter shaft, threading each conductor through the catheter shaft, and then sliding the electrodes into position along the catheter shaft. While various proposals have been made to simplify this process, they are each fraught with problems. For example, one possible technique that could possibly be used is laser direct structuring (“LDS”), which is a process during which a computer-controlled laser beam travels over a plastic substrate to activate its surface at locations where the conductive path is to be situated. A critical requirement of laser direct structuring processes, however, is that the plastic substrate has a high degree of heat resistance. Although there are several polymers that could potentially satisfy this requirement, it is often problematic to melt extrude them into thin-walled substrates having a three-dimensional shape (e.g., tubular), which is needed for catheters and many other types of products.

As such, a need currently exists for a polymer composition that can be readily melt-extruded and circuitized by a laser direct structuring process.

### SUMMARY OF THE INVENTION

In accordance with one embodiment of the present invention, a melt-extruded substrate is disclosed that comprises a polymer composition that includes a thermotropic liquid crystalline polymer and a laser activatable additive. The polymer composition has a melt viscosity of from about 60 to about 250 Pa·s, as determined in accordance with ISO Test No. 11443 at 15° C. higher than the melting temperature of the composition and at a shear rate of 1000 seconds⁻¹.

In accordance with another embodiment of the present invention, a medical article is disclosed that comprises a polymer composition that includes a thermotropic liquid crystalline polymer and a laser activatable additive, wherein the polymer composition has a melt viscosity of from about 60 to about 250 Pa·s, as determined in accordance with ISO Test No. 11443 at 15° C. higher than the melting temperature of the composition and at a shear rate of 1000 seconds⁻¹.

Other features and aspects of the present invention are set forth in greater detail below.

### BRIEF DESCRIPTION OF THE FIGURE

A full and enabling disclosure of the present invention, including the best mode thereof to one skilled in the art, is set forth more particularly in the remainder of the specification, including reference to the accompanying figures, in which:

FIG. 1 is a perspective view of one embodiment of a catheter circuit that may employ the melt-extruded substrate of the present invention; and

FIG. 2 is a front cross-sectional view of the circuit of FIG. 1.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to a polymer composition that can be readily melt-extruded into a shaped three-dimensional substrate (e.g., tube) and also applied with a conductive element using a laser direct structuring (“LDS”) process. In this regard, the composition contains a thermotropic liquid crystalline polymer and a laser activatable additive. The specific nature of the polymer and relative concentration of the polymer and additive are selectively controlled so that the resulting composition can possess a relatively high melt viscosity, which allows the resulting substrate to better maintain its shape during extrusion. The polymer composition, for instance, typically has a melt viscosity of from about 60 to about 250 Pa·s, in some embodiments from about 70 to about 200 Pa·s, and in some embodiments, from about 80 to about 180 Pa·s, determined at a shear rate of 1000 seconds⁻¹. Of course, in certain embodiments, other melt viscosities may be employed, such as those from about 40 to about 80 Pa·s. Melt viscosity may be determined in accordance with ISO Test No. 11443 at 15° C. higher than the melting temperature of the composition.

The melt strength of the polymer composition may also be relatively high, which can be characterized by the engineering stress and/or viscosity at a certain percent strain and at the melting temperature of the composition. As explained in more detail below, such testing may be performed in accordance with the ARES-EVF during which an extensional viscosity fixture (“EVF”) is used on a rotational rheometer to allow the measurement of the material stress versus percent strain. In this regard, the present inventors have discovered that the polymer composition can have a relatively high maximum engineering stress even at relatively high percent strains. For example, the composition can exhibit its maximum engineering stress at a percent strain of from about 0.3% to about 1.5%, in some embodiments from about 0.4% to about 1.5%, and in some embodiments, from about 0.6% to about 1.2%. The maximum engineering stress may, for instance, range from about 340 kPa to about 600 kPa, in some embodiments from about 350 kPa to about 500 kPa, and in some embodiments, from about 370 kPa to about 420 kPa. Just as an example, at a percent strain of about 0.6%, the composition can exhibit a relatively high engineering stress of 340 kPa to about 600 kPa, in some embodiments from about 350 kPa to about 500 kPa, and in some embodiments, from about 360 kPa to about 400 kPa. The elongational viscosity may also range from about 350 kPa·s to about 1500 kPa·s, in some embodiments from about 500 kPa·s to about 1000 kPa·s, and in some embodiments, from about 600 kPa·s to about 900 kPa·s. Without intending to be limited by theory, the ability to achieve enhanced such an increased melt strength can allow the resulting substrate to better maintain its shape during melt extrusion without exhibiting a substantial amount of sag.

Various embodiments of the present invention will now be described in further detail.

### I. Polymer Composition

A. Liquid Crystalline Polymer

The liquid crystalline polymer that is employed in the polymer composition is generally classified as “thermotropic” to the extent that they can possess a rod-like structure and exhibit a crystalline behavior in its molten state (e.g., thermotropic nematic state). Such polymers may be formed from one or more types of repeating units as is known in the art. The liquid crystalline polymer may, for example, contain one or more aromatic ester repeating units, typically in an amount of from about 60 mol. % to about 99.9 mol. %, in some embodiments from about 70 mol. % to about 99.5 mol. %, and in some embodiments, from about 80 mol. % to about 99 mol. % of the polymer. The aromatic ester repeating units may be generally represented by the following Formula (I):

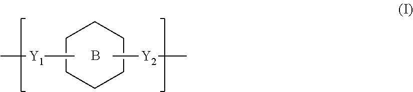

wherein,

ring B is a substituted or unsubstituted 6-membered aryl group (e.g., 1,4-phenylene or 1,3-phenylene), a substituted or unsubstituted 6-membered aryl group fused to a substituted or unsubstituted 5- or 6-membered aryl group (e.g., 2,6-naphthalene), or a substituted or unsubstituted 6-membered aryl group linked to a substituted or unsubstituted 5- or 6-membered aryl group (e.g., 4,4-biphenylene); and

Y₁ and Y₂ are independently O, C(O), NH, C(O)HN, or NHC(O).

Typically, at least one of Y₁ and Y₂ are C(O). Examples of such aromatic ester repeating units may include, for instance, aromatic dicarboxylic repeating units (Y₁ and Y₂ in Formula I are C(O)), aromatic hydroxycarboxylic repeating units (Y₁ is O and Y₂ is C(O) in Formula I), as we as various combinations thereof.

Aromatic dicarboxylic repeating units, for instance, may be employed that are derived from aromatic dicarboxylic acids, such as terephthalic acid, isophthalic acid, 2,6-naphthalenedicarboxylic acid, diphenyl ether-4,4′-dicarboxylic acid, 1,6-naphthalenedicarboxylic acid, 2,7-naphthalenedicarboxylic acid, 4,4′-dicarboxybiphenyl, bis(4-carboxyphenyl)ether, bis(4-carboxyphenyl)butane, bis(4-carboxyphenyl)ethane, bis(3-carboxyphenyl)ether, bis(3-carboxyphenyl)ethane, etc., as well as alkyl, alkoxy, aryl and halogen substituents thereof, and combinations thereof. Particularly suitable aromatic dicarboxylic acids may include, for instance, terephthalic acid (“TA”), isophthalic acid (“IA”), and 2,6-naphthalenedicarboxylic acid (“NDA”). When employed, repeating units derived from aromatic dicarboxylic acids (e.g., IA, TA, and/or NDA) typically constitute from about 5 mol. % to about 60 mol. %, in some embodiments from about 10 mol. % to about 55 mol. %, and in some embodiments, from about 15 mol. % to about 50% of the polymer.

Aromatic hydroxycarboxylic repeating units may also be employed that are derived from aromatic hydroxycarboxylic acids, such as, 4-hydroxybenzoic acid; 4-hydroxy-4′-biphenylcarboxylic acid; 2-hydroxy-6-naphthoic acid; 2-hydroxy-5-naphthoic acid; 3-hydroxy-2-naphthoic acid; 2-hydroxy-3-naphthoic acid; 4′-hydroxyphenyl-4-benzoic acid; 3′-hydroxyphenyl-4-benzoic acid; 4′-hydroxyphenyl-3-benzoic acid, etc., as well as alkyl, alkoxy, aryl and halogen substituents thereof, and combination thereof. Particularly suitable aromatic hydroxycarboxylic acids are 4-hydroxybenzoic acid (“HBA”) and 6-hydroxy-2-naphthoic acid (“HNA”). When employed, repeating units derived from hydroxycarboxylic acids (e.g., HBA and/or HNA) typically constitute from about 10 mol. % to about 85 mol. %, in some embodiments from about 20 mol. % to about 80 mol. %, and in some embodiments, from about 25 mol. % to about 75% of the polymer.

Other repeating units may also be employed. In certain embodiments, for instance, repeating units may be employed that are derived from aromatic diols, such as hydroquinone, resorcinol, 2,6-dihydroxynaphthalene, 2,7-dihydroxynaphthalene, 1,6-dihydroxynaphthalene, 4,4′-dihydroxybiphenyl (or 4,4′-biphenol), 3,3′-dihydroxybiphenyl, 3,4′-dihydroxybiphenyl, 4,4′-dihydroxybiphenyl ether, bis(4-hydroxyphenyl)ethane, etc., as well as alkyl, alkoxy, aryl and halogen substituents thereof, and combinations thereof. Particularly suitable aromatic diols may include, for instance, hydroquinone (“HQ”) and 4,4′-biphenol (“BP”). When employed, repeating units derived from aromatic diols (e.g., HQ and/or BP) typically constitute from about 1 mol. % to about 30 mol. %, in some embodiments from about 2 mol. % to about 25 mol. %, and in some embodiments, from about 5 mol. % to about 20% of the polymer. Repeating units may also be employed, such as those derived from aromatic amides (e.g., acetaminophen (“APAP”)) and/or aromatic amines (e.g., 4-aminophenol (“AP”), 3-aminophenol, 1,4-phenylenediamine, 1,3-phenylenediamine, etc.). When employed, repeating units derived from aromatic amides (e.g., APAP) and/or aromatic amines (e.g., AP) typically constitute from about 0.1 mol. % to about 20 mol. %, in some embodiments from about 0.5 mol. % to about 15 mol. %, and in some embodiments, from about 1 mol. % to about 10% of the polymer. It should also be understood that various other monomeric repeating units may be incorporated into the polymer. For instance, in certain embodiments, the polymer may contain one or more repeating units derived from non-aromatic monomers, such as aliphatic or cycloaliphatic hydroxycarboxylic acids, dicarboxylic acids, dials, amides, amines, etc. Of course, in other embodiments, the polymer may be “wholly aromatic” in that it lacks repeating units derived from non-aromatic (e.g., aliphatic or cycloaliphatic) monomers.

Although not necessarily required, the liquid crystalline polymer may be “low naphthenic” to the extent that they contain a minimal content of repeating units derived from naphthenic hydroxycarboxylic acids and naphthenic dicarboxylic acids, such as naphthalene-2,6-dicarboxylic acid (“NDA”), 6-hydroxy-2-naphthoic acid (“HNA”), or combinations thereof. That is, the total amount of repeating units derived from naphthenic hydroxycarboxylic and/or dicarboxylic acids (e.g., NDA, HNA, or a combination of F-INA and NDA) is typically no more than 30 mol. %, in some embodiments no more than about 15 mol. %, in some embodiments no more than about 10 mol. %, in some embodiments no more than about 8 mol. %, and in some embodiments, from 0 mol. % to about 5 mol. % of the polymer (e.g., 0 mol. %). Despite the absence of a high level of conventional naphthenic acids, it is believed that the resulting “low naphthenic” polymer is still capable of exhibiting good thermal and mechanical properties.

In one particular embodiment, for example, the polymer may be formed from repeating units derived from 4-hydroxybenzoic acid (“HBA”) and terephthalic acid (“TA”) and/or isophthalic acid (“IA”), as well as various other optional constituents. The repeating units derived from 4-hydroxybenzoic acid (“HBA”) may constitute from about 10 mol. % to about 80 mol. %, in some embodiments from about 30 mol. % to about 75 mol. %, and in some embodiments, from about 45 mol. % to about 70 mol. % of the polymer. The repeating units derived from terephthalic acid (“TA”) and/or isophthalic acid (“IA”) may likewise constitute from about 5 mol. % to about 40 mol. %, in some embodiments from about 10 mol. % to about 35 mol. %, and in some embodiments, from about 15 mol. % to about 35 mol. % of the polymer. Repeating units may also be employed that are derived from 4,4′-biphenol (“BP”) and/or hydroquinone (“HQ”) in an amount from about 1 mol. % to about 30 mol. %, in some embodiments from about 2 mol. % to about 25 mol. %, and in some embodiments, from about 5 mol. % to about 20 mol. % of the polymer. Other possible repeating units may include those derived from 6-hydroxy-2-naphthoic acid (“HNA”), 2,6-naphthalenedicarboxylic acid (“NDA”), and/or acetaminophen (“APAP”). For example, repeating units derived from HNA, NDA, and/or APAP may each constitute from about 1 mol. % to about 35 mol. %, in some embodiments from about 2 mol. % to about 30 mol. %, and in some embodiments, from about 3 mol. % to about 25 mol. % when employed.

Liquid crystalline polymers may be prepared by initially introducing the aromatic monomer(s) used to form ester repeating units (e.g., aromatic hydroxycarboxylic acid, aromatic dicarboxylic acid, etc.) and/or other repeating units (e.g., aromatic diol, aromatic amide, aromatic amine, etc.) into a reactor vessel to initiate a polycondensation reaction. The particular conditions and steps employed in such reactions are well known, and may be described in more detail in U.S. Pat. No. 4,161,470 to Calundann; U.S. Pat. No. 5,616,680 to Linstid, Ill, et al.; U.S. Pat. No. 6,114,492 to Linstid, Ill, et al.; U.S. Pat. No. 6,514,611 to Shepherd, et al.; and WO 2004/058851 to Waggoner. The vessel employed for the reaction is not especially limited, although it is typically desired to employ one that is commonly used in reactions of high viscosity fluids. Examples of such a reaction vessel may include a stirring tank-type apparatus that has an agitator with a variably-shaped stirring blade, such as an anchor type, multistage type, spiral-ribbon type, screw shaft type, etc., or a modified shape thereof. Further examples of such a reaction vessel may include a mixing apparatus commonly used in resin kneading, such as a kneader, a roll mill, a Banbury mixer, etc.

If desired, the reaction may proceed through the acetylation of the monomers as known the art. This may be accomplished by adding an acetylating agent (e.g., acetic anhydride) to the monomers. Acetylation is generally initiated at temperatures of about 90° C. During the initial stage of the acetylation, reflux may be employed to maintain vapor phase temperature below the point at which acetic acid byproduct and anhydride begin to distill. Temperatures during acetylation typically range from between 90° C. to 150° C., and in some embodiments, from about 110° C. to about 150° C. If reflux is used, the vapor phase temperature typically exceeds the boiling point of acetic acid, but remains low enough to retain residual acetic anhydride. For example, acetic anhydride vaporizes at temperatures of about 140° C. Thus, providing the reactor with a vapor phase reflux at a temperature of from about 110° C. to about 130° C. is particularly desirable. To ensure substantially complete reaction, an excess amount of acetic anhydride may be employed. The amount of excess anhydride will vary depending upon the particular acetylation conditions employed, including the presence or absence of reflux. The use of an excess of from about 1 to about 10 mole percent of acetic anhydride, based on the total moles of reactant hydroxyl groups present is not uncommon.

Acetylation may occur in in a separate reactor vessel, or it may occur in situ within the polymerization reactor vessel. When separate reactor vessels are employed, one or more of the monomers may be introduced to the acetylation reactor and subsequently transferred to the polymerization reactor. Likewise, one or more of the monomers may also be directly introduced to the reactor vessel without undergoing pre-acetylation.

In addition to the monomers and optional acetylating agents, other components may also be included within the reaction mixture to help facilitate polymerization. For instance, a catalyst may be optionally employed, such as metal salt catalysts (e.g., magnesium acetate, tin(I) acetate, tetrabutyl titanate, lead acetate, sodium acetate, potassium acetate, etc.) and organic compound catalysts (e.g., N-methylimidazole). Such catalysts are typically used in amounts of from about 50 to about 500 parts per million based on the total weight of the recurring unit precursors. When separate reactors are employed, it is typically desired to apply the catalyst to the acetylation reactor rather than the polymerization reactor, although this is by no means a requirement.

The reaction mixture is generally heated to an elevated temperature within the polymerization reactor vessel to initiate melt polycondensation of the reactants. Polycondensation may occur, for instance, within a temperature range of from about 300° C. to about 400° C. For instance, one suitable technique for forming the liquid crystalline polymer may include charging precursor monomers and acetic anhydride into the reactor, heating the mixture to a temperature of from about 90° C. to about 150° C. to acetylize a hydroxyl group of the monomers (e.g., forming acetoxy), and then increasing the temperature to from about 300° C. to about 400° C. to carry out melt polycondensation. As the final polymerization temperatures are approached, volatile byproducts of the reaction (e.g., acetic acid) may also be removed so that the desired molecular weight may be readily achieved. The reaction mixture is generally subjected to agitation during polymerization to ensure good heat and mass transfer, and in turn, good material homogeneity. The rotational velocity of the agitator may vary during the course of the reaction, but typically ranges from about 10 to about 100 revolutions per minute (“rpm”), and in some embodiments, from about 20 to about 80 rpm. To build molecular weight in the melt, the polymerization reaction may also be conducted under vacuum, the application of which facilitates the removal of volatiles formed during the final stages of polycondensation. The vacuum may be created by the application of a suctional pressure, such as within the range of from about 5 to about 30 pounds per square inch (“psi”), and in some embodiments, from about 10 to about 20 psi.

Following melt polymerization, the molten polymer may be discharged from the reactor, typically through an extrusion orifice fitted with a die of desired configuration, cooled, and collected. Commonly, the melt is discharged through a perforated die to form strands that are taken up in a water bath, pelletized and dried. In some embodiments, the melt polymerized polymer may also be subjected to a subsequent solid-state polymerization method to further increase its molecular weight. Solid-state polymerization may be conducted in the presence of a gas (e.g., air, inert gas, etc.). Suitable inert gases may include, for instance, include nitrogen, helium, argon, neon, krypton, xenon, etc., as well as combinations thereof. The solid-state polymerization reactor vessel can be of virtually any design that will allow the polymer to be maintained at the desired solid-state polymerization temperature for the desired residence time. Examples of such vessels can be those that have a fixed bed, static bed, moving bed, fluidized bed, etc. The temperature at which solid-state polymerization is performed may vary, but is typically within a range of from about 250° C. to about 350° C. The polymerization time will of course vary based on the temperature and target molecular weight. In most cases, however, the solid-state polymerization time will be from about 2 to about 12 hours, and in some embodiments, from about 4 to about 10 hours.

The resulting liquid crystalline polymer typically has a high molecular weight as is reflected by its melt viscosity. That is, the melt viscosity may range from about 20 to about 200 Pa·s, in some embodiments from about 40 to about 180 Pa·s, and in some embodiments, from about 50 to about 150 Pa·s at a shear rate of 1000 seconds⁻¹. Melt viscosity may be determined in accordance with ISO Test No. 11443 at 15° C. higher than the melting temperature of the composition. The melting temperature of the liquid crystalline polymer may likewise range from about 300° C. to about 400° C., in some embodiments from about 310° C. to about 395° C., and in some embodiments, from about 320° C. to about 380° C. The melting temperature may be determined as is well known in the art using differential scanning calorimetry (“DSC”), such as determined by ISO Test No, 11357,

B. Laser Activatable Additive

The polymer composition of the present invention is “laser activatable” in the sense that it contains an additive that is activated by a laser direct structuring (“LDS”) process. In such a process, the additive is exposed to a laser that causes the release of metals. The laser thus draws the pattern of conductive elements onto the part and leaves behind a roughened surface containing embedded metal particles. These particles act as nuclei for the crystal growth during a subsequent plating process (e.g., copper plating, gold plating, nickel plating, silver plating, zinc plating, tin plating, etc). Laser activatable additives typically constitute from about 0.1 wt. % to about 30 wt. %, in some embodiments from about 0.5 wt. % to about 20 wt. %, and in some embodiments, from about 1 wt. % to about 10 wt. % of the polymer composition. Likewise, liquid crystalline polymers typically constitute from about 20 wt. % to about 80 wt. %, in some embodiments from about 30 wt. % to about 75 wt. %, and in some embodiments, from about 40 wt. % to about 70 wt. % of the polymer composition.

The laser activatable additive generally includes spinel crystals, which may include two or more metal oxide cluster configurations within a definable crystal formation. For example, the overall crystal formation may have the following general formula:

AB₂O₄

wherein,

A is a metal cation having a valance of 2, such as cadmium, chromium, manganese, nickel, zinc, copper, cobalt, iron, magnesium, tin, titanium, etc., as well as combinations thereof; and

B is a metal cation having a valance of 3, such as chromium, iron, aluminum, nickel, manganese, tin, etc., as well as combinations thereof.

Typically, A in the formula above provides the primary cation component of a first metal oxide cluster and B provides the primary cation component of a second metal oxide cluster. These oxide clusters may have the same or different structures. In one embodiment, for example, the first metal oxide cluster has a tetrahedral structure and the second metal oxide cluster has an octahedral cluster. Regardless, the clusters may together provide a singular identifiable crystal type structure having heightened susceptibility to electromagnetic radiation. Examples of suitable spinel crystals include, for instance, MgAl₂O₄, ZnAl₂O₄, FeAl₂O₄, CuFe₂O₄, CuCr₂O₄, MnFe₂O₄, NiFe₂O₄, TiFe₂O₄, FeCr₂O₄, MgCr₂O₄, etc. Copper chromium oxide (CuCr₂O₄) is particularly suitable for use in the present invention and is available from Shepherd Color Co. under the designation “Shepherd Black 1GM.”

C. Optional Additives

If desired, the composition may optionally contain one or more additives if so desired, such as fillers, flow aids, antimicrobials, pigments, antioxidants, stabilizers, surfactants, waxes, solid solvents, flame retardants, anti-drip additives, and other materials added to enhance properties and processability. For example, a filler material may be incorporated into the polymer composition to enhance strength. Mineral fillers may, for instance, be employed in the polymer composition to help achieve the desired mechanical properties and/or appearance. Such fillers are particularly desirable when forming thermoformed substrates. When employed, mineral fillers typically constitute from about 5 wt. % to about 60 wt. %, in some embodiments from about 10 wt. % to about 55 wt. %, and in some embodiments, from about 20 wt. % to about 50 wt. % of the polymer composition. Clay minerals may be particularly suitable for use in the present invention. Examples of such clay minerals include, for instance, talc (Mg₃Si₄O₁₀(OH)₂), halloysite (Al₂Si₂O₅(OH)₄), kaolinite (Al₂Si₂O₅(OH)₄), illite ((K,H₃O)(Al,Mg,Fe)₂(Si,Al)₄O₁₀[(OH)₂(H₂O)]), montmorillonite (Na,Ca)_{0.33}(Al,Mg)₂Si₄O₁₀(OH)₂.nH₂O), vermiculite ((MgFe,Al)₃(Al,Si)₄O₁₀(OH)₂. 4H₂O), palygorskite ((Mg,Al)₂Si₄O₁₀(OH).4(H₂O)), pyrophyllite (Al₂Si₄O₁₀(OH)₂), etc., as well as combinations thereof. In lieu of, or in addition to, clay minerals, still other mineral fillers may also be employed. For example, other suitable silicate fillers may also be employed, such as calcium silicate, aluminum silicate, mica, diatomaceous earth, wollastonite, and so forth. Mica, for instance, may be particularly suitable. There are several chemically distinct mica species with considerable variance in geologic occurrence, but all have essentially the same crystal structure. As used herein, the term “mica” is meant to generically include any of these species, such as muscovite (KAl₂(AlSi₃)O₁₀(OH)₂), biotite (K(Mg,Fe)₃(AlSi₃)O₁₀(OH)₂), phlogopite (KMg₃(AlSi₃)O₁₀(OH)₂), lepidolite (K(Li,Al)₂₋₃(AlSi₃)O₁₀(OH)₂), glauconite (K,Na)(Al,Mg,Fe)₂(Si,Al)₄O₁₀(OH)₂, etc. as well as combinations thereof.

Fibers may also be employed as a filler material to further improve the mechanical properties. Such fibers generally have a high degree of tensile strength relative to their mass. For example, the ultimate tensile strength of the fibers (determined in accordance with ASTM D2101) is typically from about 1,000 to about 15,000 Megapascals (“MPa”), in some embodiments from about 2,000 MPa to about 10,000 MPa, and in some embodiments, from about 3,000 MPa to about 6,000 MPa. The high strength fibers may be formed from materials that are also generally insulative in nature, such as glass, ceramics (e.g., alumina or silica), aramids (e.g., Kevlar® marketed by E. I. Du Pont de Nemours, Wilmington, Del.), polyolefins, polyesters, etc., as well as mixtures thereof. Glass fibers are particularly suitable, such as E-glass, A-glass, C-glass, D-glass, AR-glass, R-glass, S1-glass, S2-glass, etc., and mixtures thereof.

The volume average length of the fibers may be from about 50 to about 400 micrometers, in some embodiments from about 80 to about 250 micrometers, in some embodiments from about 100 to about 200 micrometers, and in some embodiments, from about 110 to about 180 micrometers. The fibers may also have a narrow length distribution. That is, at least about 70% by volume of the fibers, in some embodiments at least about 80% by volume of the fibers, and in some embodiments, at least about 90% by volume of the fibers have a length within the range of from about 50 to about 400 micrometers, in some embodiments from about 80 to about 250 micrometers, in some embodiments from about 100 to about 200 micrometers, and in some embodiments, from about 110 to about 180 micrometers. The fibers may also have a relatively high aspect ratio (average length divided by nominal diameter) to help improve the mechanical properties of the resulting polymer composition. For example, the fibers may have an aspect ratio of from about 2 to about 50, in some embodiments from about 4 to about 40, and in some embodiments, from about 5 to about 20 are particularly beneficial. The fibers may, for example, have a nominal diameter of about 10 to about 35 micrometers, and in some embodiments, from about 15 to about 30 micrometers. The relative amount of the fibers in the polymer composition may also be selectively controlled to help achieve the desired mechanical properties without adversely impacting other properties of the composition, such as its flowability. For example, the fibers may constitute from about 2 wt. % to about 40 wt. %, in some embodiments from about 5 wt. % to about 35 wt. %, and in some embodiments, from about 6 wt. % to about 30 wt. % of the polymer composition.

Still other additives that can be included in the composition may include, for instance, antimicrobials, pigments (e.g., carbon black), antioxidants, stabilizers, surfactants, waxes, solid solvents, and other materials added to enhance properties and processability. Lubricants, for instance, may be employed in the polymer composition. Examples of such lubricants include fatty acids esters, the salts thereof, esters, fatty acid amides, organic phosphate esters, and hydrocarbon waxes of the type commonly used as lubricants in the processing of engineering plastic materials, including mixtures thereof. Suitable fatty acids typically have a backbone carbon chain of from about 12 to about 60 carbon atoms, such as myristic acid, palmitic acid, stearic acid, arachic acid, montanic acid, octadecinic acid, parinric acid, and so forth. Suitable esters include fatty acid esters, fatty alcohol esters, wax esters, glycerol esters, glycol esters and complex esters. Fatty acid amides include fatty primary amides, fatty secondary amides, methylene and ethylene bisamides and alkanolamides such as, for example, palmitic acid amide, stearic acid amide, oleic acid amide, N,N′-ethylenebisstearamide and so forth. Also suitable are the metal salts of fatty acids such as calcium stearate, zinc stearate, magnesium stearate, and so forth; hydrocarbon waxes, including paraffin waxes, polyolefin and oxidized polyolefin waxes, and microcrystalline waxes. Particularly suitable lubricants are acids, salts, or amides of stearic acid, such as pentaerythritol tetrastearate, calcium stearate, or N,g-ethylenebisstearamide. When employed, the lubricant(s) typically constitute from about 0.05 wt. % to about 1.5 wt. %, and in some embodiments, from about 0.1 wt. % to about 0.5 wt. % (by weight) of the polymer composition.

The resulting polymer composition may have a relatively high melting temperature. For example, the melting temperature of the polymer composition may be from about 300° C. to about 400° C., in some embodiments from about 310° C. to about 395° C., and in some embodiments, from about 320° C. to about 380° C. Even at such melting temperatures, the ratio of the deflection temperature under load (“DTUL”), a measure of short term heat resistance, to the melting temperature may still remain relatively high. For example, the ratio may range from about 0.67 to about 1.00, in some embodiments from about 0.68 to about 0.95, and in some embodiments, from about 0.70 to about 0.85. The specific DTUL values may, for instance, range from about 200° C. to about 350° C., in some embodiments from about 210° C. to about 320° C., and in some embodiments, from about 220° C. to about 290° C.

The polymer composition may also have a solidification rate and/or crystallization rate that allows for extruding without producing tears, ruptures, stress fractures, blisters, etc. In this regard, the polymer composition may have a relatively high heat of crystallization, such as about 3.3 J/g or more, in some embodiments about 3.5 J/g or more, in some embodiments from about 3.5 to about 10 J/g, and in some embodiments, from about 3.7 to about 6.0 J/g. As used herein, the heat of crystallization is determined according to ISO Test No. 11357. The polymer composition may also possess a relatively high degree of heat resistance. For example, the composition may possess a “blister free temperature” of about 250° C. or greater, in some embodiments about 260° C. or greater, in some embodiments from about 265° C. to about 320° C., and in some embodiments, from about 270° C. to about 300° C. As explained in more detail below, the “blister free temperature” is the maximum temperature at which a substrate does not exhibit blistering when placed in a heated silicone oil bath. Such blisters generally form when the vapor pressure of trapped moisture exceeds the strength of the substrate, thereby leading to delamination and surface defects.

### II. Melt-Extruded Substrates

Any of a variety of melt extrusion techniques may generally be employed to form substrates in accordance with the present invention. Suitable melt extrusion techniques may include, for instance, tubular trapped bubble film processes, flat or tube cast film processes, slit die flat cast film processes, etc. The resulting substrate may have a variety of different forms, such as sheets, films, tubes, etc. Regardless, the substrate is typically thin in nature and thus, for instance, has a thickness of about 10 millimeters or less, in some embodiments from about 0.01 to about 8 millimeters, in some embodiments from about 0.05 to about 6 millimeters, and in some embodiments, from about 0.1 to about 2 millimeters. Conductive elements may be formed on the substrate using a laser direct structuring process (“LDS”). Activation with a laser causes a physio-chemical reaction in which the spinel crystals are cracked open to release metal atoms. These metal atoms can act as a nuclei for metallization (e.g., reductive copper coating). The laser also creates a microscopically irregular surface and ablates the polymer matrix, creating numerous microscopic pits and undercuts in which the copper can be anchored during metallization.

Due to its unique properties, the melt-extruded substrate of the present invention may be employed in a wide variety of different products. For example, in certain embodiments, the substrate can be employed in a medical article, such as a device, instrument, apparatus, implant, etc., which can be used to examine, diagnose, prevent, and/or treat disease or other conditions. One example of such a medical article is a catheter that can be used to examine, diagnose, and/or treat a patient while it is positioned at a specific location within a body. Such catheters are commonly inserted into a vessel near the surface of the body and guided to a specific location within the body. For example, one procedure often referred to as “catheter ablation” employs a catheter to convey an electrical stimulus to a selected location within the human body to create tissue necrosis. Another procedure often referred to as “mapping” employs a catheter with sensing electrodes to monitor various forms of electrical activity in the human body. Due to the unique thermal properties and heat resistance provided by the polymer composition of the present invention, it may be beneficially employed to form melt-extruded substrates of a generally tubular shape, as are typically employed in such catheters. Furthermore, due to its ability to be laser activated, conductive elements can be readily formed thereon to help provide the desired electrical stimulus or sensing functionality.

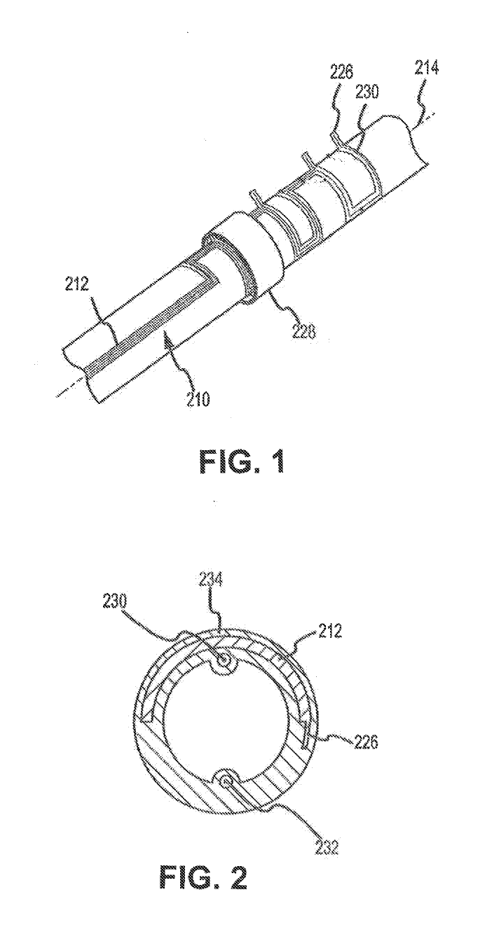

Referring to FIGS. 1-2, for example, one particular embodiment of a circuit 210 is shown that can be employed in a catheter as is known in the art, such as for use in connection with a mapping or ablation catheter and/or another tubular object configured for insertion into a body cavity or blood vessel. The circuit 210 includes a substrate 212 having a longitudinal axis 214 for extending along at least a portion of the length of the catheter in which it is employed. The substrate 212 is generally tubular-shaped in that at least a portion of it is curved when it is embedded within a catheter shaft. In certain embodiments, the substrate 212 may be melt-extruded and formed from the laser activatable polymer composition of the present invention. In this regard, conductive elements 230 (“traces”) can be formed on a surface of the substrate 212 through laser activation, such as described above. The proximal end of the conductive elements may 230 may terminate at a solder pad compatible with a circuit connector (e.g., zif type connector) and the distal end may terminate at or near a pad 226. The pad 226 is connected to an electrode 228 provided on the catheter for ablation or mapping. For example, the electrode 228 may emit an electrical stimulus to create tissue necrosis and/or the electrode 228 may include a sensing electrode to monitor various forms of electrical activity in the human body. The electrode 228 may be connected to the pad 226 in a variety of ways, such as by welding, conductive adhesives, etc.

After assembly of the circuitized substrate 212, the entire catheter shaft can be encapsulated with a reflow material 234 to seal and/or secure the placement of the circuit and electrodes. The electrodes may be bonded and/or adhered to the shaft during the reflow process. The reflow material 234 may be a polymeric material, such as a liquid crystalline polymer, polyimide, polyamide, etc. To help maintain electrical integrity and avoid shaft disruption, pull wires may also be disposed adjacent to the substrate 212. For example, as shown in FIG. 2, a first pull wire 230 and a second pull wire 232 may be disposed adjacent to the substrate 212. If desired, an external tubing (not shown) may also be employed to allow for some movement of the circuit within the catheter to help prevent bucking when the catheter is deflected or compressed. Such external tubing may, for instance, be formed from polytetrafluroethylene (“PTFE”) and may be disposed between the circuit 210 and the material of the shaft of the catheter. Although not shown, the catheter may further include a tip configured for tissue ablation.

Apart from those referenced above, the melt-extruded substrate of the present invention may also be employed in a wide variety of other components, such as desktop computers, portable electronic components, etc. Examples of suitable portable electronic components include cellular telephones, laptop computers, small portable computers (e.g., ultraportable computers, netbook computers, and tablet computers), wrist-watch devices, pendant devices, headphone and earpiece devices, media players with wireless communications capabilities, handheld computers (also sometimes called personal digital assistants), remote controllers, global positioning system (GPS) devices, handheld gaming devices, etc.

The present invention may be better understood with reference to the following examples.

### Test Methods

Melt Viscosity: The melt viscosity (Pa·s) may be determined in accordance with ISO Test No, 11443 at a shear rate of 1000 s⁻¹ and temperature 15° C. above the melting temperature (e.g., 350° C. or 375° C.) using a Dynisco LCR7001 capillary rheometer. The rheometer orifice (die) had a diameter of 1 mm, length of 20 mm, L/D ratio of 20.1, and an entrance angle of 180°. The diameter of the barrel was 9.55 mm+0.005 mm and the length of the rod was 233.4 mm.

Melting Temperature: The melting temperature (“Tm”) may be determined by differential scanning calorimetry (“DSC”) as is known in the art. The melting temperature is the differential scanning calorimetry (DSC) peak melt temperature as determined by ISO Test No. 11357. Under the DSC procedure, samples may be heated and cooled at 20° C. per minute as stated in ISO Standard 10350 using DSC measurements conducted on a TA Q2000 Instrument.

Melt Elongation: Melt elongation properties (i.e., stress, strain, and elongational viscosity) may be determined in accordance with the ARES-EVF: Option for Measuring Extensional Velocity of Polymer Melts, A. Franck, which is incorporated herein by reference. In this test, an extensional viscosity fixture (“EVF”) is used on a rotational rheometer to allow the measurement of the engineering stress at a certain percent strain. More particularly, a thin rectangular polymer melt sample is adhered to two parallel cylinders: one cylinder rotates to wind up the polymer melt and lead to continuous uniaxial deformation in the sample, and the other cylinder measures the stress from the sample. An exponential increase in the sample length occurs with a rotating cylinder. Therefore, the Hencky strain (ε_{H}) is determined as function of time by the following equation: ε_{H}(t)=In(L(t)/Lₒ), where Lₒ is the initial gauge length of and L(t) is the gauge length as a function of time. The Hencky strain is also referred to as percent strain. Likewise, the elongational viscosity is determined by dividing the normal stress (kPa) by the elongation rate (s⁻¹). Specimens tested according to this procedure have a width of 1.27 mm, length of 30 mm, and thickness of 0.8 mm. The test may be conducted at the melting temperature (e.g., about 360° C.) and elongation rate of 2 s⁻¹.

Deflection Under Load Temperature (“DTUC”): The deflection under load temperature may be determined in accordance with ISO Test No. 75-2 (technically equivalent to ASTM D648-07). More particularly, a sample having a length of 80 mm, thickness of 10 mm, and width of 4 mm may be subjected to an edgewise three-point bending test in which the specified load is 1.8 MPa. The specimen may be lowered into a silicone oil bath where the temperature is raised at 2° C. per minute until it deflects 0.25 mm (0.32 mm for ISO Test No. 75-2).

Blister Free Temperature: To test blister resistance, a 127×12.7×0.8 mm test substrate is formed at 5° C. to 10° C. higher than the melting temperature of the polymer resin, as determined by DSC. Ten (10) substrates are immersed in a silicone oil at a given temperature for 3 minutes, subsequently removed, cooled to ambient conditions, and then inspected for blisters (i.e., surface deformations) that may have formed. The test temperature of the silicone oil begins at 250° C. and is increased at 10° C. increments until a blister is observed on one or more of the test substrates. The “blister free temperature” for a tested material is defined as the highest temperature at which all ten (10) bars tested exhibit no blisters. A higher blister free temperature suggests a higher degree of heat resistance.

Tensile Modulus, Tensile Stress, and Tensile Elongation: Tensile properties are tested according to ISO Test No. 527 (technically equivalent to ASTM D638). Modulus and strength measurements are made on the same test strip sample having a length of 80 mm, thickness of 10 mm, and width of 4 mm. The testing temperature is 23° C., and the testing speeds are 1 or 5 mm/min.

Flexural Modulus, Flexural Stress, and Flexural Strain: Flexural properties are tested according to ISO Test No. 178 (technically equivalent to ASTM D790). This test is performed on a 64 mm support span. Tests are run on the center portions of uncut ISO 3167 multi-purpose bars. The testing temperature is 23° C. and the testing speed is 2 mm/min.

Izod Notched impact Strength: Notched Izod properties are tested according to ISO Test No. 180 (technically equivalent to ASTM D256, Method A). This test is run using a Type A notch. Specimens are cut from the center of a multi-purpose bar using a single tooth milling machine. The testing temperature is 23° C.

### EXAMPLE

Three (3) samples (Samples 1-3) of a liquid crystalline polymer are melt-polymerized from 4-hydroxybenzoic acid (“HBA”), 2,6-hydroxynaphthoic acid (“HNA”), terephthalic acid (“TA”), 4,4′-biphenol (“BP”), and acetaminophen (“APAP”), such as described in U.S. Pat. No. 5,508,374 to Lee, et al. The naphthenic content is 5 mol. %. The melt-polymerized polymer is solid-state polymerized until melt viscosities of 62, 98, and 133 Pa·s (at 1000 s⁻¹) for Samples 1-3, respectively, are achieved.

Once formed, compositions are formed that contain 41.5 wt. % of the polymer, 38.5 wt. % talc, and 20.0 wt. % of a laser activatable additive concentrate. The concentrate is formed from 30 wt. % Shepherd 1GM (CuCr₂O₇) and 70 wt. % of a liquid crystalline polymer (melt viscosity of about 90 Pa·s at 1000 s⁻¹) such as described above. A twin screw extruder is used to form the polymer compositions. The laser activatable concentrate and the polymer (dried to below 100 ppm moisture) are added in barrel #1, while talc is added downstream therefrom. Vacuum is applied to the compounded melt prior to exit from the extruder to remove any volatiles. The compositions are extruded into strands and quenched in water bath prior to pelletization. The temperature setting of the extruder barrels is between 300 to 350° C. and a screw speed between 250 and 800 rpm is used depending on the intensity of shear from the screw design.

The compounding conditions and resulting melt properties for the samples are summarized in the table below.

| | Compounded Sample | | |
|---|---|---|---|
| | 1 | 2 | 3 |
| Melt Viscosity (Pa-s) | 111 | 136 | 162 |
| (350° C., 1000 s⁻¹) | | | |
| Melting Temperature (° C.) | 341 | 342 | 341 |
| Throughput Rate (lb/hr) | 230 | 230 | 230 |
| Screw Speed (RPM) | 425 | 425 | 425 |
| Extrudate Temp. (° C.) | 370 | 374 | 378 |
| Vacuum (″Hg) | 29 | 29 | 29 |
| Barrel Zone Temp. Setting (° C.) | 300 to 340 | 300 to 340 | 300 to 340 |
| Die Temp. Setting (° C.) | 355 | 355 | 355 |

The samples are then molded into a part for various strength and thermal testing as indicated above. The results are set forth below.

| | Sample | | |
|---|---|---|---|
| | 1 | 2 | 3 |
| Tensile Strength (MPa) | 106 | 108 | 108 |
| Elongation at Break (%) | 3.0 | 3.1 | 3.2 |
| Tensile Modulus (MPa) | 10,560 | 10,634 | 10,578 |
| Flexural Strength (MPa) | 130 | 129 | 130 |
| Flex Strain (%) | 3.1 | 3.0 | 3.1 |
| Flexural Modulus (MPa) | 12,351 | 12,673 | 12,619 |
| Notched Izod (kJ/m²) | 5 | 6 | 7 |
| DTUL @ 1.8 MPa (° C.) | 229 | 235 | 236 |
| Blister Free Temp. (° C.) | 280 | 280 | 280 |

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A melt-extruded substrate comprising a polymer composition that includes a thermotropic liquid crystalline polymer and a laser activatable additive, wherein the polymer composition has a melt viscosity of from about 60 to about 250 Pa·s, as determined in accordance with ISO Test No. 11443 at 15° C. higher than the melting temperature of the composition and at a shear rate of 1000 seconds⁻¹.

2. The melt-extruded substrate of claim 1, wherein the polymer composition has a melt viscosity of from about 70 to about 200 Pa·s, as determined in accordance with ISO Test No. 11443 at 15° C. higher than the melting temperature of the composition and at a shear rate of 1000 seconds⁻¹.

3. The melt-extruded substrate of claim 1, wherein the composition exhibits a maximum engineering stress of from about 340 kPa to about 600 kPa, as determined at the melting temperature of the composition with an extensional viscosity fixture and a rotational rheometer.

4. The melt-extruded substrate of claim 1, wherein the polymer composition exhibits a maximum engineering stress at a percent strain of from about 0.3% to about 1.5%, as determined at the melting temperature of the composition with an extensional viscosity fixture and a rotational rheometer.

5. The melt-extruded substrate of claim 1, wherein the polymer composition exhibits an elongational viscosity of from about 350 kPa·s to about 1500 kPa·s, as determined at the melting temperature of the composition with an extensional viscosity fixture and a rotational rheometer.

6. The melt-extruded substrate of claim 1, wherein the melting temperature of the composition is from about 300° C. to about 400° C.

7. The melt-extruded substrate of claim 1, wherein the thermotropic liquid crystalline polymer contains aromatic ester repeating units, the aromatic ester repeating units including aromatic dicarboxylic acid repeating units and aromatic hydroxycarboxylic acid repeating units.

8. The melt-extruded substrate of claim 7, wherein the aromatic hydroxycarboxylic acid repeating units are derived from 4-hydroxybenzoic acid, 6-hydroxy-2-naphthoic acid, or a combination thereof and/or the aromatic dicarboxylic acid repeating units are derived from terephthalic acid, isophthalic acid, or a combination thereof.

9. The melt-extruded substrate of claim 8, wherein the thermotropic liquid crystalline polymer further contains hydroquinone, 4,4′-biphenol, or a combination thereof.

10. The melt-extruded substrate of claim 7, wherein the liquid crystalline polymer is formed from repeating units derived from 4-hydroxybenzoic acid in an amount from about 10 mol. % to about 80 mol. %, repeating units derived from terephthalic acid and/or isophthalic acid in an amount from about 5 mol. % to about 40 mol. %, and repeating units derived from 4,4° -biphenol and/or hydroquinone in an amount from about 1 mol. % to about 30 mol. %.

11. The melt-extruded substrate of claim 1, wherein the laser activatable additive includes a spinel crystal.

12. The melt-extruded substrate of claim 11, wherein the crystal has the following general formula:
AB₂O4
wherein,
A is a metal cation having a valance of 2; and
B is a metal cation having a valance of 3.

13. The melt-extruded substrate of claim 12, wherein the spinel crystal is MgAl₂O₄, ZnAl₂O₄, FeAl₂O₄, CuFe₂O₄, CuCr₂O₄, MnFe₂O₄, NiFe₂O₄, TiFe₂O₄, FeCr₂O₄, MgCr₂O₄, or a combination thereof.

14. The melt-extruded substrate of claim 1, wherein laser activatable additives constitute from about 0.1 wt. % to about 30 wt. % of the polymer composition and liquid crystalline polymers constitute from about 20 wt. % to about 80 wt. % of the polymer composition.

15. The melt-extruded substrate of claim 1, wherein the polymer composition comprises a mineral filler.

16. The melt-extruded substrate of claim 1, wherein the substrate has a generally tubular shape.

17. A circuit comprising conductive elements disposed on a surface of a melt-extruded substrate, wherein the melt-extruded substrate comprises a polymer composition that includes a thermotropic liquid crystalline polymer and a laser activatable additive, wherein the polymer composition has a melt viscosity of from about 60 to about 250 Pa·s, as determined in accordance with ISO Test No. 11443 at 15° C. higher than the melting temperature of the composition and at a shear rate of 1000 seconds⁻¹.

18. A medical article comprising a polymer composition that includes a thermotropic liquid crystalline polymer and a laser activatable additive, wherein the polymer composition has a melt viscosity of from about 60 to about 250 Pa·s, as determined in accordance with ISO Test No. 11443 at 15° C. higher than the melting temperature of the composition and at a shear rate of 1000 seconds⁻¹.

19. The medical article of claim 18, wherein the medical article includes a catheter.

20. The medical article of claim 18, wherein the substrate has a longitudinal axis that extends along at least a portion of the length of the catheter, and wherein the substrate is embedded within a shaft.
